(19) United States
(12) Patent Application Publication
(10) Pub. No.: US 2013/0253333 A1
(43) Pub. Date: Sep. 26, 2013

(54) TISSUE INTERFACE ELEMENTS FOR APPLICATION OF OPTICAL SIGNALS INTO TISSUE OF A PATIENT

(71) Applicants: Sarah Hayman, Boulder, CO (US); Friso Schlottau, Lyons, CO (US); Paulo E. X. Silveira, Boulder, CO (US); Aaron Wegner, Longmont, CO (US)

(72) Inventors: Sarah Hayman, Boulder, CO (US); Friso Schlottau, Lyons, CO (US); Paulo E. X. Silveira, Boulder, CO (US); Aaron Wegner, Longmont, CO (US)

(73) Assignee: Nellcor Puritan Bennett LLC, Boulder, CO (US)

(21) Appl. No.: 13/427,455

(22) Filed: Mar. 22, 2012

(51) Int. Cl. A61B 6/08

(52) U.S. Cl. 600/476

(57) ABSTRACT

Systems and methods for applying optical signals into tissue of a patient are provided herein. In one example, a tissue interface pad for applying an optical signal to tissue of a patient is provided. The tissue interface pad includes a first surface configured to interface with the tissue of the patient, at least one guide channel disposed within the tissue interface pad and configured to route an input optical fiber carrying the optical signal to a first location in the tissue interface pad, and a second surface at the first location configured to direct the optical signal from the input optical fiber into the tissue through the first surface.

### TECHNICAL FIELD

Aspects of the disclosure are related to the field of medical devices, and in particular, tissue interface elements for application of optical signals into tissue of a patient and optical measurement of physiological parameters of blood and tissue.

### TECHNICAL BACKGROUND

Various devices, such as pulse oximetry devices or photon density wave (PDW) devices, can measure parameters of blood or tissue in a patient, such as heart rate and oxygen saturation of hemoglobin, among other parameters. These devices are non-invasive measurement devices, typically employing solid-state lighting elements, such as light-emitting diodes (LEDs) or solid state lasers, to introduce light into the tissue of a patient. The light is then detected and analyzed to determine the parameters of the blood flow in the patient.

In some examples, optical fibers are employed to transfer optical signals between processing and signaling equipment and the tissue of a patient. These optical fibers can often deliver higher quality signals to the tissue than directly introducing optical signals from a light source onto the tissue, in part because optical fibers allow the placement of the optical source (or multiple sources) away from the tissue, enabling the use of higher-quality sources without substantially impacting cost. However, consistent application and detection of the light or other optical signals into the tissue of the patient can be difficult to achieve using optical fibers, especially in examples where long optical fibers are employed. For example, challenges are encountered with introducing optical signals into tissue from an optical fiber routed parallel to the tissue, due in part to the large minimum bend radius of optical fibers. These challenges are accentuated when using optical fibers with a large core radius. Such large core fibers are capable of collecting more light from tissue, the fiber light collection capacity being approximately proportional to the area of the fiber core. However, the larger the core diameter of the fiber requires a larger bend radius.

In further examples, measurement and processing systems are located remotely from various optical elements used for interfacing optical signals with the tissue of the patient. This configuration can provide some patient mobility and ease of use for the clinical staff by using a flexible fiber optic cable between the equipment.

### OVERVIEW

Systems and methods for applying optical signals into tissue of a patient are provided herein. In a first example, a tissue interface pad for applying an optical signal to tissue of a patient is provided. The tissue interface pad includes a first surface configured to interface with the tissue of the patient, at least one guide channel disposed within the tissue interface pad and configured to route an input optical fiber carrying the optical signal to a first location in the tissue interface pad, and a second surface at the first location configured to direct the optical signal from the input optical fiber into the tissue through the first surface.

In a second example, a tissue interface pad for applying an optical signal to tissue of a patient is provided. The tissue interface pad includes a first surface configured to interface with the tissue of the patient, at least one guide channel disposed within the tissue interface pad and configured to route an input optical fiber carrying the optical signal to a first location in the tissue interface pad, and a second surface configured to reflect the optical signal received from the input optical fiber through an optically transmissive portion of the tissue interface pad to direct the optical signal into the tissue through the optically transmissive portion of the tissue interface pad and the first surface.

In a third example, a tissue interface pad for applying an optical signal to tissue of a patient is provided. The tissue interface pad includes a first surface configured to interface with the tissue of the patient, at least one guide channel disposed within the tissue interface pad and configured to route an input optical fiber carrying the optical signal to a first location in the tissue interface pad, and an optical interface element coupled to one end of the input optical fiber at the first location and configured to direct the optical signal received from the input optical fiber toward a second surface. The second surface is configured to reflect the optical signal to direct the optical signal into the tissue through the optical interface element and the first surface.

This Overview is provided to introduce a selection of concepts in a simplified form that are further described below in the Detailed Description. It should be understood that this Overview is not intended to identify key features or essential features of the claimed subject matter, nor is it intended to be used to limit the scope of the claimed subject matter.

### BRIEF DESCRIPTION OF THE DRAWINGS

FIG. 1 is a system diagram illustrating a system for applying optical signals to tissue of a patient.

FIG. 2 is a flow diagram illustrating a method of operation of a system for applying optical signals to tissue of a patient.

FIG. 3 is a system diagram illustrating a system for applying optical signals to tissue of a patient.

FIG. 4 is a system diagram illustrating a system for applying optical signals to tissue of a patient.

FIG. 5 is a system diagram illustrating a system for applying optical signals to tissue of a patient.

FIG. 6 is a system diagram illustrating a system for applying optical signals to tissue of a patient.

### DETAILED DESCRIPTION

Various physiological parameters of tissue and blood of a patient can be determined non-invasively, such as optically (e.g., optical spectroscopy). A first example is the use of light at dual wavelengths to perform non-invasive arterial blood oxygen saturation measurements, as is done in pulse oximetry. In another example, optical signals introduced into the tissue of the patient are modulated according to a high-frequency modulation signal to create a photon density wave (PDW) optical signal in the tissue undergoing measurement. Due to the interaction between the tissue or blood and the PDW optical signal, various characteristics of the PDW optical signal can be affected, such as through scattering or propagation by various components of the tissue and blood. The various physiological parameters can include any parameter associated with the blood or tissue of the patient, such as regional oxygen saturation (rSO2), arterial oxygen saturation (Sp02), heart rate, lipid concentrations, among other parameters, including combinations thereof. Another type of optical tissue interaction is a photoacoustic-based interaction, where the light launched into the tissue is absorbed by an appropriate absorber, which causes localized heating and expansion of the absorber and results in acoustic waves that can be detected by an appropriate acoustic sensor.

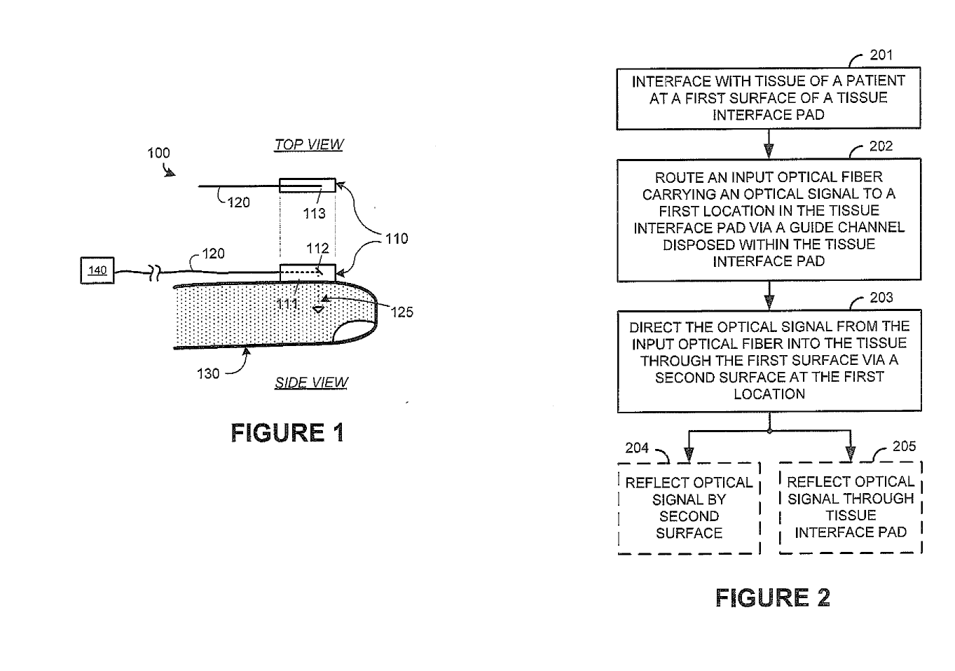

As a first example of a system for measuring a physiological parameter of blood in a patient, FIG. 1 is presented. FIG. 1 illustrates system 100, which includes tissue interface pad 110, input optical fiber 120, tissue 130, and measurement system 140. Tissue interface pad 110 includes first surface 111 and second surface 112. In operation, optical signals generated by measurement system 140 are applied to tissue 130 for measurement of a physiological parameter, as indicated by optical signal 125. In this example, optical signal 125 is applied to tissue 130 via input optical fiber 120 and tissue interface pad 110. Only the tissue interface pad is shown in the top view in FIG. 1 to highlight the tissue interface pad, it should be understood the tissue 130 may have been included. Also, although only one optical fiber 120 is shown in FIG. 1, in typical examples more than one optical fiber is employed in a parallel configuration to optical fiber 120. However, the examples shown herein focus on the tissue interface pad and simplify the optical fiber quantity for clarity.

FIG. 2 is a flow diagram illustrating a method of operation of system 100 for applying optical signals to tissue of a patient. The operations of FIG. 2 are referenced herein parenthetically. In FIG. 2, tissue interface pad 110 interfaces (201) with tissue 130 of a patient at first surface 111 of tissue interface pad 110. First surface 111 couples to biological tissue, namely tissue 130, to allow for introduction of optical signals into tissue 130. In further examples, first surface 111 also allows for detection of optical signals propagated through tissue 130. Tissue interface pad 110 routes (202) input optical fiber 120 carrying optical signal 125 to first location 113 in tissue interface pad 110 via a guide channel disposed within tissue interface pad 110. The guide channel can include a groove or channel which holds input optical fiber 120 and terminates an end of input optical fiber 120 at first location 113.

Tissue interface pad 110 directs (203) optical signal 125 from input optical fiber 120 into tissue 130 through first surface 111 via second surface 112 at first location 113. Optical signal 125 is received from input optical fiber 120 and reflected or refracted by second surface 112 for eventual introduction into tissue 130. Different reflection or refraction configurations can be employed for second surface 112, as discussed below. Advantageously, optical fiber 120 can be placed horizontally along tissue 130 while optical signal 125 can be introduced vertically into tissue 130 via tissue interface pad 110.

In a first example configuration of second surface 112, tissue interface pad 110 reflects (204) optical signal 125 using second surface 112 to direct optical signal 125 through first surface 111 and into tissue 130. Further optical interface elements can be employed to optically couple input optical fiber 120 to second surface 112 and to tissue 130. For example, a prism can be configured to mate with an end of input optical fiber 120 and couple optical signal 125 into tissue 130. This prism may be composed of an optically transparent material, such as optical adhesive, plastic, or glass, and may be optically index matched to a material of input optical fiber 120 to allow propagation of optical signal 125 from input optical fiber 120 into tissue 130.

In a second example configuration of second surface 112, tissue interface pad 110 reflects (205) optical signal 125 through tissue interface pad 110 itself to direct optical signal through first surface 111 and into tissue 130. In this second example configuration, at least a portion of tissue interface pad 110 is composed of optically transmissive material and optical signal 125 is directed through this optically transmissive material after being reflected by second surface 112. Tissue interface pad 110 can be made of a clear or optically transparent material in this second example configuration. This optically transparent material can be optically index matched to the material of input optical fiber 120 to allow propagation of optical signal 125 from input optical fiber 120 through tissue interface pad 110 and into tissue 130.

Although only input optical fiber 120 is shown for simplicity in FIG. 1, further optical fibers or links may be included to receive optical signals which have been propagated, reflected, or scattered by tissue 130. Upon receiving optical signals after propagation through tissue 130, measurement system 140 may process the detected optical signals to determine various characteristics of the detected optical signals. Physiological parameters of the tissue and patient can then be identified based on the various characteristics of the detected optical signals.

Referring back to FIG. 1, tissue interface pad 110 comprises a physical structure having a first surface that couples to biological tissue, namely tissue 130. The first surface includes at least one optical signal emission point and may include at least one optical signal detection point. Tissue interface pad 110 includes a mechanical arrangement to position and hold optical fiber 120 in a generally parallel arrangement to tissue 130. These mechanical arrangements can include grooves, c-grooves, v-grooves, channels, holes, snap-fit features, or other elements to route input optical fiber 120 to a desired position in tissue interface pad 110. As shown in FIG. 1, tissue interface pad 110 positions an end of input optical fiber 120 at location 113. Tissue interface pad 110 may be comprised of plastic, foam, rubber, glass, metal, adhesive, or some other material, including combinations thereof. In some examples, tissue interface pad 110 is comprised of optically transmissive materials, such as optically transmissive adhesive, optically transmissive plastic, glass, acrylic glass, polymethyl methacrylate (PMMA), or other materials, including combinations thereof.

Measurement system 140 includes optical interfaces, digital processors, computer systems, microprocessors, circuitry, non-transient computer-readable media, user interfaces, or other processing devices or software systems, and may be distributed among multiple processing devices. Measurement system 140 may also include photon density wave (PDW) generation and measurement equipment, electrical to optical conversion circuitry and equipment, optical modulation equipment, and optical waveguide interface equipment. Measurement system 140 also includes light emitting elements, such as LEDs, laser diodes, solid-state lasers, or other light emitting devices and combinations thereof, along with associated driving circuitry. Optical couplers, cabling, or attachments can be included to optically mate light emitting elements to input optical fiber 120.

Tissue 130 is shown in FIG. 1 as a finger of a patient. It should be understood that tissue 130 can be any tissue portion of a patient, such as a finger, toe, arm, leg, earlobe, forehead, or other tissue portion of a patient. In this example, tissue 130 is a portion of the tissue of a patient undergoing measurement of a physiological blood parameter. The wavelength of signals applied to the tissue can be selected based on many factors, such as optimized to a wavelength strongly absorbed by hemoglobin, lipids, proteins, water, or other tissue and blood components of tissue 130.

Optical fiber 120 comprises an optical waveguide, and uses glass, polymer, air, space, or some other material as the transport media for transmission of light, and can include multimode fiber (MMF) or single mode fiber (SMF) materials. A sheath or loom can be employed to bundle optical fiber 120 together with further optical links for convenience. One end of optical fiber 120 mates with an associated optical driver component of measurement system 140, and the other end of optical fiber 120 is configured to terminate in tissue interface pad 110 for optically interfacing with tissue 130. Various optical interfacing elements discussed herein can be employed to optically couple optical signals carried by optical fiber 120 to tissue 130. Optical fiber 120 may include many different signals sharing the same associated link, as represented by the associated line in FIG. 1, comprising channels, forward links, reverse links, user communications, overhead communications, frequencies, wavelengths, phases, modulation frequencies, modulation depths, carriers, timeslots, spreading codes, logical transportation links, packets, or communication directions.

Also, although FIG. 1 illustrates only a single optical fiber 120, it should be understood that any number of input links and measurement links can be included, as well as any associated optical source and detector equipment. For example, tissue interface pad 110 may route many optical fibers to different physical locations on tissue 130, and these optical fibers can carry optical signals of different wavelengths. Alternatively, or in addition, tissue interface pad 110 may have measurement links positioned at different distances from input links or positioned over different anatomical structures.

The term ‘optical’ or ‘light’ is used herein for convenience. It should be understood that the applied and detected signals are not limited to visible light, and can comprise any photonic, electromagnetic, or energy signals, such as visible, infrared, ultraviolet, radio, x-ray, gamma, or other signals. Additionally, the use of optical fibers or optical cables herein is merely representative of a waveguide used for propagating signals between a transceiver and tissue of a patient. Suitable waveguides would be employed for different electromagnetic signal types.

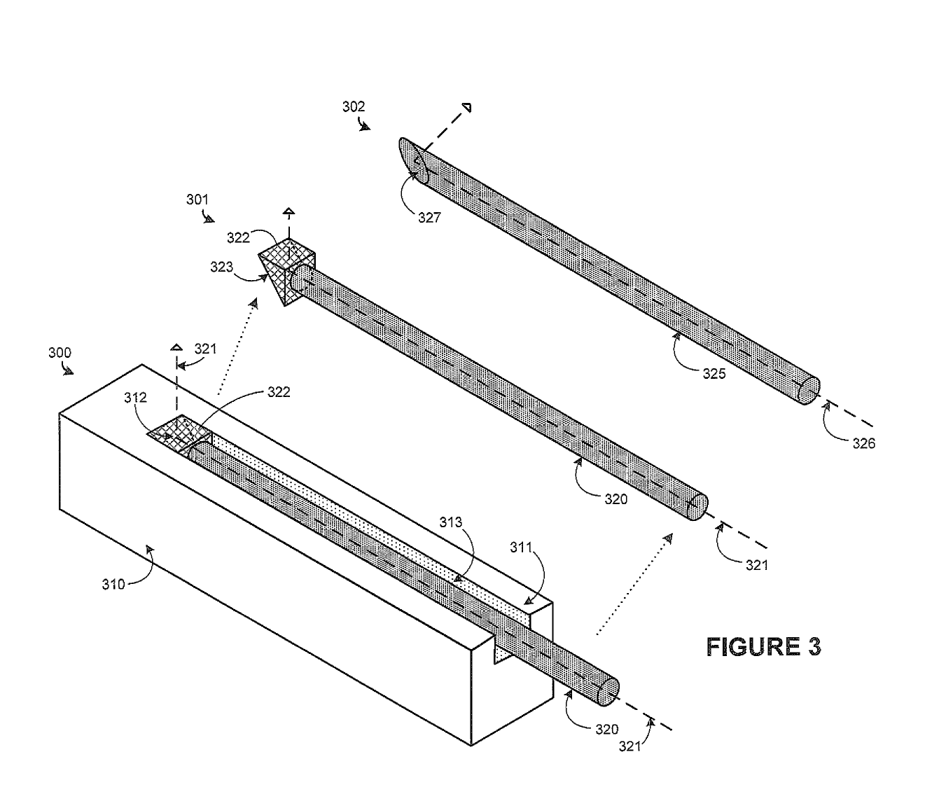

FIG. 3 is a system diagram illustrating an oblique view of system 300 for applying optical signals to tissue of a patient. System 300 is an example of system 100, although system 100 may use different configurations. System 300 includes tissue interface pad 310, optical fiber 320, and optical interface element 322. A detailed view of the assembly comprising optical fiber 320 and optical interface element 322 is shown in view 301. Another embodiment of elements of system 300 is shown in view 302, namely modified optical fiber 325. Although not required, modified optical fiber 302 can be employed in system 300 instead of the assembly shown in view 301.

Tissue interface pad 310 may be composed of plastic, foam, rubber, glass, metal, adhesive, or some other material, including combinations thereof. Tissue interface pad 310 includes first surface 311, second surface 312, and channel 313. First surface 311 forms a generally planar surface as shown on the ‘top’ of tissue interface pad 310 in FIG. 3. First surface 311 is configured to interface with tissue of a patient, not shown in FIG. 3 for clarity, to allow for introduction of optical signals into the tissue.

Second surface 312 comprises an angled surface formed from the material of tissue interface pad 310 at one end of channel 313. In some examples, second surface 312 is coated with a reflective coating. In other examples, such as when tissue interface pad 310 is composed of a metallic material, second surface 312 is polished to create a reflective surface. The angle of second surface 312 is typically 45 degrees with respect to first surface 311, or 135 degrees with respect to the longitudinal axis shared by optical fiber 320 and channel 313. Thus, when optical signals 321 carried by optical fiber 320 are incident upon second surface 312, then optical signals 321 are directed generally perpendicular to the longitudinal axis shared by optical fiber 320 and channel 313 and through the plane of first surface 311 into the tissue under measurement. In further examples, different angles of second surface 312 can be employed based on the refractive index of the tissue interface pad elements, such as second surface 312 or optical interface element 322, or to increase reflective efficiency when using higher numerical aperture types of optical fibers.

Channel 313 is formed into tissue interface pad 310 to hold optical fiber 320. In this example, channel 313 cuts through the plane formed by first surface 311. Other configurations of channel 313 can be employed such as a bore-hole that does not break the plane formed by first surface 311 and sized to hold optical fiber 320. Also, channel 313 is shown as a generally square groove (in cross-section). Other groove cross-sectional styles can be employed, such as c-grooves, v-grooves, or snap-fit features, among other styles. Channel 313 is typically sized to fit optical fiber 320 securely or tightly. In further configurations, channel 313 can be coated or painted with an optically absorbent or opaque material to prevent optical signals escaping from the length of optical fiber 320 from entering the tissue or other optical fibers if included.

Also included in system 300 is optical interface element 322. Optical interface element 322 is configured to optically couple optical signals 321 carried by optical fiber 320 to second surface 312 and into the tissue under measurement. In this example, one end of optical fiber 320 is mated to optical interface element 322 by abutting optical interface element 322 with a generally flat end. More specifically, this end of optical fiber 320 is typically cut and polished perpendicular to the longitudinal axis of optical fiber 320. Optical interface element 322 is typically composed of a material that is index matched to the material of optical fiber 320, such as being composed of a material with a similar index of refraction as optical fiber 320. Thus, optical signals 321 will generally not be refracted when exiting optical fiber 320 until emerging off of second surface 312. Once optical signals 321 are refracted by second surface 312, optical signals 321 are directed into the tissue under measurement through first surface 311.

Optical interface element 322 can comprise a prism composed of glass, plastic, or other optically transmissive material. In some examples, optical interface element 322 is composed of an optical adhesive which is introduced into a void or chamber adjacent to second surface 312. This optical adhesive, once cured, will then form an index matched transition prism between optical fiber 320, second surface 312, and the tissue under measurement. As shown in view 301, optical interface element 322 has an angled surface 323 which mates with second surface 312. In some examples, such as when optical interface element 322 comprises a glass or plastic prism, angled surface 323 is coated with a reflective material and forms second surface 312 for reflecting optical signals 321 into the tissue. However, in examples where optical interface element 322 comprises optical adhesive introduced in a liquid state and cured into a void adjacent to second surface 312, then second surface 312 is typically made reflective by the introduction of optical adhesive and thus angled surface 323 would merely be the surface of optical interface element 322 which is mated to second surface 312. Surface 323 can be made reflective by using a metallic mirror (e.g. protected silver), which is less reflective but operates over a wide range of wavelengths, or using a dielectric mirror (e.g. a stack of dielectric layers of controlled indices of refraction and thicknesses) which is more reflective at specific wavelengths, among other techniques and materials.

In an alternate embodiment, modified optical fiber 325 is shown in view 302. Modified optical fiber 325 can be composed of a similar material as optical fiber 320. However, modified optical fiber 325 is shown with an obtusely angled surface 327 located at one end of optical fiber 325 to form a hypotenuse surface. Surface 327 is then polished and typically coated with an optically reflective material. When optical signals 326 are carried by optical fiber 325 and are incident upon surface 327, optical signals 326 are reflected by surface 327 and directed generally perpendicular to the longitudinal axis of optical fiber 325. If optical fiber 325 is placed into channel 313 of tissue interface pad 310, then the angled surface 327 can act as second surface 312 to direct optical signals 326 into tissue under measurement. It should be noted that surface 327 does not have to be coated in all examples. For example, total internal reflection (TIR) techniques can direct the optical signals into the tissue up to a specific critical angle. Most glasses and polymers of indexes around n=1.4-1.5 can properly direct the optical signals using 45 degree cuts in the optical fiber (respect to an associated surface of a tissue interface pad) without coating. TIR can fail if foreign material, such as dirt, oil, or moisture, touches the TIR interface, and thus a reflective coating can be employed to make the system more robust. Other optical beam turners can be employed, such as diffraction elements.

The reflective coating employed by either second surface 312 or angled surface 327 can comprise aluminum, silver, dichroic stack, or other materials, including combinations thereof. The optical adhesive employed to form optical interface element 322 or to bond optical interface element 322 to an end of optical fiber 320 can comprise Loctite 3321 or Norland 68 compositions which are cured using ultraviolet (UV) light. Other optically transmissive adhesives can be employed, including combinations thereof.

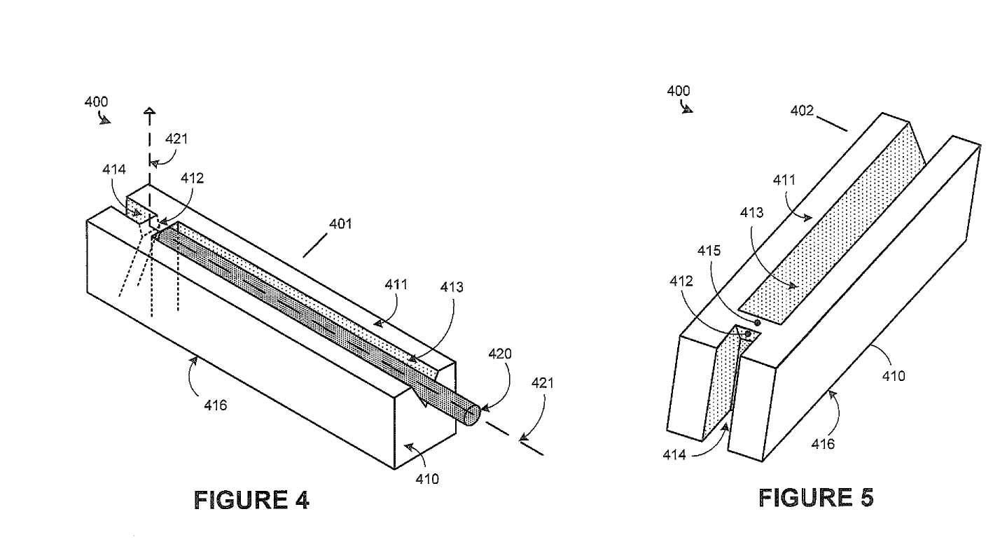

FIGS. 4 and 5 are system diagrams illustrating oblique views of system 400 for applying optical signals to tissue of a patient. System 400 is an example of system 100, although system 100 may use different configurations. System 400 includes tissue interface pad 410 and optical fiber 420. A first view 401 of system 400 shows optical fiber 420 inserted in tissue interface pad 410. A second view 402 of system 400 omits optical fiber 420 for clarity. The hidden lines forming elements of tissue interface pad 410, such as those in FIG. 4 defining portions of second surface 412, transmissive material 415, or optical index gap 414 are included to highlight some portions of the hidden structure due to the limitations of the oblique view. It is not intended that these lines are exact mechanical representations of the internal wireframe structure, nor do the internal structures necessarily terminate as the dashed lines indicate.

Tissue interface pad 410 includes first surface 411, second surface 412, groove 413, optical index gap 414, transmissive material 415, and back surface 416. Tissue interface pad 410 may be composed of an optically transmissive material, and is typically an index matched material to that of optical fiber 420 to prevent reflection or refraction of optical signal 421 at an optical interface of optical fiber 420 and tissue interface 410. In some examples, tissue interface pad 410 is comprised of optically transmissive adhesive, optically transmissive plastic, glass, acrylic glass, polymethyl methacrylate (PMMA), or other materials, including combinations thereof. Thus, optical signals 321 will generally not be refracted when exiting optical fiber 420 until reflecting off of second surface 412. In some examples, only a portion of tissue interface pad 410 is composed of an optically transmissive material, such as elements 412 and 415 and any further portions intended to carry optical signal 421. First surface 411 forms a generally planar surface as shown on the ‘top’ of tissue interface pad 410 in FIGS. 4 and 5. First surface 411 is configured to interface with tissue of a patient, not shown in FIGS. 4 and 5 for clarity, to allow for introduction of optical signals into the tissue.

Second surface 412 comprises an angled surface formed from the material of tissue interface pad 410 near one end of groove 413. In this example, second surface 412 is separated from an end of optical fiber 420 by a small amount of optically transmissive material 415 of tissue interface pad 410. Second surface 412 is also adjacent to optical index gap 414. Optical index gap 414 provides a change in optical index to refract optical signal 421 once optical signal 421 is carried through material 415 of tissue interface pad 410. Thus, when optical signal 421 is transferred from optical fiber 420 through material 415, second surface 412 will refract optical signal 421 through further optically transmissive material of tissue interface pad 410. This refraction is shown in FIG. 4 as optical signal 421 directed ‘upward’ by second surface 412 through tissue interface pad 410 and first surface 411.

Optical index gap 414 can comprise an air-material interface between material of tissue interface pad 410 and the surrounding air. It may be desirable to ensure moisture or other foreign material is not introduced into optical index gap 414, and protective cladding material can be coated onto surfaces of optical index gap 414 or filled into optical index gap 414 to ensure a desired index of refraction for second surface 412 is maintained. In some examples, a cladding material of lower refractive index is employed in optical index gap 414 to alter the angle required for second surface 412. In typical examples, the angle of second surface 412 is 45 degrees with respect to first surface 411 of tissue interface pad 410, or 135 degrees with respect to the longitudinal axis of optical fiber 420. Thus, when optical signal 421 carried by optical fiber 420 are incident upon second surface 412, then optical signal 421 is directed generally perpendicular to the longitudinal axis shared by optical fiber 420 and groove 413 and through the plane of first surface 411 into the tissue under measurement. If a lower optical index cladding material is used in optical index gap 414 than that of the material of tissue interface pad 410, a different angle of second surface 412 can be employed to direct optical signal 421 generally perpendicular to the longitudinal axis of optical fiber 420.

Optical fiber 420 mates with material 415 at a generally flat surface of material 415. Thus, the end of optical fiber 420 which mates with tissue interface pad 410 is cut and polished generally perpendicular to the longitudinal axis of optical fiber 420. An optical adhesive can be employed to optically mate the one end of optical fiber 420 to the flat surface of material 415. Groove 413 is formed into tissue interface pad 410 to hold optical fiber 420. In this example, groove 413 cuts through the plane formed by first surface 411. Other configurations of channel 413 can be employed such as a bore-hole that does not break the plane formed by first surface 411 and sized to hold optical fiber 420. Also, groove 413 is shown as a generally v-shaped groove (in cross-section). The v-groove configuration of groove 413 ensures side-to-side self-alignment of optical fiber 420 within groove 413. Other groove cross-sectional styles can be employed, such as c-grooves, square grooves, or snap-fit features, among other styles. Groove 413 is typically sized to fit optical fiber 420 securely or tightly. In further configurations, groove 413 can be coated or painted with an optically absorbent or opaque material to prevent optical signals leaking from the length of optical fiber 420 from entering the tissue or other optical fibers if included. Optical fiber 420 can be fastened into groove 413 by an adhesive along the length of optical fiber 420 or by a cover piece or jacket coupled over first surface 411 of tissue interface pad 410. If a cover piece is employed, and optically opaque material can be used to prevent stray signals from leaving tissue interface pad 410 or from entering into optical fiber 420.

Although this example illustrates second surface 412 directing optical signal 421 ‘upward’ through material 415 of tissue interface pad 410, other configurations can be employed. For example, second surface 412 can be angled oppositely than as shown in FIGS. 4 and 5, thus making the angle of second surface 412 as 135 degrees with respect to first surface 411, or 45 degrees with respect to the longitudinal axis of optical fiber 420. Thus, when optical signal 421 carried by optical fiber 420 are incident upon second surface 412, then optical signal 421 is directed generally perpendicular and to the longitudinal axis shared by optical fiber 420 and groove 413 and ‘downward’ through the plane of back surface 416. Tissue under measurement can then be coupled to back surface 416 for measurement and application of optical signal 421 which now passes through the body of tissue interface pad 410. In these further examples, optical fiber 420 is located on an opposite side of tissue interface pad 410 than the surface which is coupled to the tissue, allowing for distance and protection for optical fiber 420 from contact with any tissue. Also, in these further examples, portions of tissue interface pad 410 which optical signals 421 pass through can be composed of an optically transmissive material, and is typically an index matched material to that of optical fiber 420 to prevent reflection or refraction of optical signal 421 at an optical interface of optical fiber 420 and tissue interface 410.

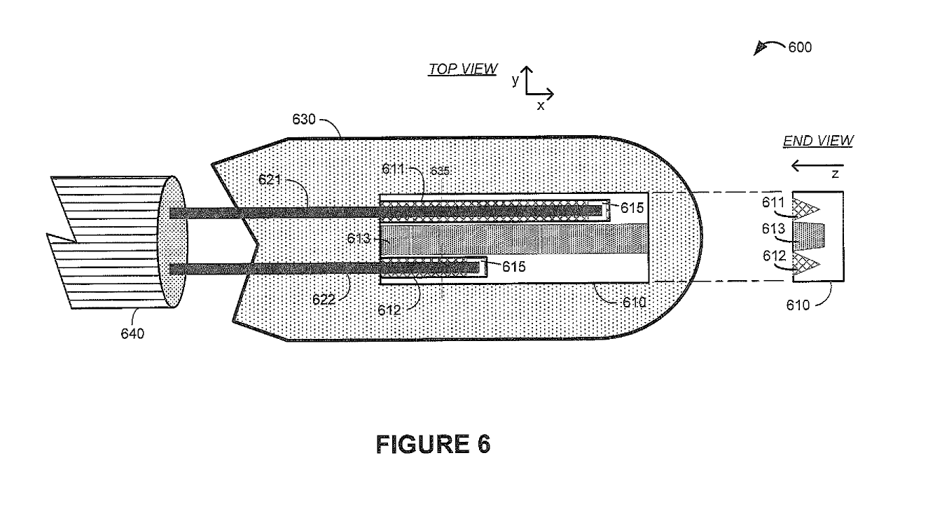

FIG. 6 is a system diagram illustrating tissue interface assembly 600. Tissue interface assembly 600 includes kayak 610 and optical cable 640. Kayak 610 is an example of tissue interface pad 110, tissue interface pad 310, or tissue interface pad 410, although these may use different configurations. Kayak 610 is coupled to tissue 630 in this example. Tissue 630 can comprise any tissue described herein, such as a finger of a patient. Optical cable 640 comprises several optical fibers, namely optical fibers 621-622, for carrying optical signals to and from kayak 610. In FIG. 6, several axes are shown for reference purposes. For the top view, a ‘y’ axis is shown relative to the ‘up-down’ page orientation and an ‘x’ axis is shown relative to the ‘left-right’ page orientation. For the end view, a axis is shown in the side view as a thickness of kayak 610. The end view is sectioned at section cut 635 from the side view. It should be understood the dashed features of FIG. 6 are merely intended to highlight various elements of system 600, and are not intended to be exact wireframe representations of the elements of system 600; variations are possible.

Kayak 610 comprises a surface for contacting tissue 630. In operation, kayak 610 will lay coincident on tissue 630. Kayak 610 also comprises two v-channels 611, 612 for routing optical fibers 621-622 to the locations shown. Each channel is positioned at a specific channel location in the ‘y’ direction, and each channel is routed to a certain length within kayak 610 in the ‘x’ direction. The depth of each channel 611-612 in the ‘z’ direction is determined by the thickness of kayak 610, and the size of each optical fiber or optical interface elements 615, among other considerations. Surfaces of kayak 610 can be colored dark to minimize optical reflection and stray light. In some examples, kayak 610 is coated or anodized to a dark color, while in other examples kayak 610 is composed of a dark material such as plastic with injected dark pigment. In yet other examples, optically transmissive portions of kayak 610 are not coated dark or composed of dark material.

In this example, optical fiber 622 is an input optical fiber for introducing optical signals into tissue 630. Output optical fiber 621 terminates at a location relative to the input optical fiber 622. Specifically, the termination point of output optical fiber 621 is located a first distance from the termination point of input optical fiber 622. Typical spacing between the input optical fiber termination point and the output optical fiber termination points are 5-10 mm for arterial-based tissue measurements, and 30-40 mm for cerebral-based tissue measurements. Advantageously, this spacing arrangement allows the optical fibers to be aligned generally parallel within kayak 610 and thus optical cable 640 is aligned along the length of tissue 630. This parallel configuration allows for greater repeatability in measurement and consistent coupling of kayak 610 to tissue 630 by reducing perpendicular or normal stresses and forces on the optical fibers and kayak 610. Although specific spacing and location dimensions are given herein, it should be understood that the dimensions may vary. Also, although tissue interface assembly 600 includes two optical fibers, a different number of optical signals and associated optical fibers can be employed.

Kayak 610 also includes optical interface elements 615. Since the optical fibers transport optical signals parallel to the surface of tissue 630, a 90 degree optical turn must be established to properly introduce the optical signals into tissue 630 or to properly detect optical signals from tissue 630. Each optical interface element 615 can comprise a prism, optical adhesive, lens, mirror, diffuser, and the like, to optically couple the associated optical fibers to the tissue under measurement. In some examples, optical interface elements 615 are formed from the material of kayak 610. The optical interface elements 615 can each be adhered to the associated optical fiber end, such as with optically transmissive glue or other adhesive.

Also included in kayak 610 is fin 613. Fin 613 is an optically opaque separation member disposed between channels 611-612 and configured to inhibit optical coupling through kayak 610 between optical fibers 621-622. Fin 613 is disposed between and generally parallel to each of channels 611-612. The material and configuration of fin 613 prevents cross-talk or inadvertent optical coupling between optical signals carried by optical fibers 621-622 as well as to prevent cross-talk or inadvertent optical coupling between optical signals introduced into and detected from tissue 630. In examples of an optically transmissive kayak, such as found in FIGS. 4-5, fin 613 can prevent interference and noise associated with multiple optical fibers carried by a single optically transmissive tissue interface pad or kayak. In examples of kayaks with even further optical fibers, further fin elements can be employed between each channel carrying an optical fiber. In further examples, a black insert is placed in the kayak between individual optical fibers, where the black insert is placed into a slit cut into the kayak between ones of the optical fibers. In yet further examples, the slit can be filled with a black epoxy.

The included descriptions and drawings depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above, but only by the claims and their equivalents.

What is claimed is:

1. A tissue interface pad for applying an optical signal to tissue of a patient, the tissue interface pad comprising:
a first surface configured to interface with the tissue of the patient;
at least one guide channel disposed within the tissue interface pad and configured to route an input optical fiber carrying the optical signal to a first location in the tissue interface pad; and
a second surface at the first location configured to direct the optical signal from the input optical fiber into the tissue through the first surface.

2. The tissue interface pad of claim 1, wherein the second surface is configured to refract the optical signal through an optically transmissive portion of the tissue interface pad to direct the optical signal from the input optical fiber into the tissue through the first surface.

3. The tissue interface pad of claim 2, wherein the optically transmissive portion of the tissue interface pad is index matched to the input optical fiber.

4. The tissue interface pad of claim 2, wherein the optically transmissive portion of the tissue interface pad is optically mated to an end of the input optical fiber at the first location with an optical adhesive.

5. The tissue interface pad of claim 1, wherein the second surface is configured to reflect the optical signal to direct the optical signal from the input optical fiber into the tissue.

6. The tissue interface pad of claim 5, further comprising:
an optical interface element coupled to the input optical fiber and configured to direct the optical signal toward the second surface.

7. The tissue interface pad of claim 6, wherein the optical interface element comprises an optical adhesive configured to optically mate an end of the input optical fiber to the second surface, and wherein the second surface comprises an optically reflective surface.

8. The tissue interface pad of claim 7, wherein the at least one guide channel comprises a chamber at the first location configured to hold the optical adhesive, and wherein the optical adhesive is disposed in the chamber to form a prism comprising the optical adhesive.

9. The tissue interface pad of claim 5, wherein the second surface comprises an optically reflective and angled surface configured to reflect the optical signal generally perpendicular to a longitudinal axis of the input optical fiber to direct the optical signal through the first surface.

10. The tissue interface pad of claim 1, further comprising:
a second guide channel disposed adjacent and parallel to the first guide channel in the tissue interface pad and configured to route a second optical fiber to a second location in the tissue interface pad; and
an optically opaque separation member disposed between the first guide channel and the second guide channel and configured to inhibit optical coupling through the tissue interface pad between the first optical fiber and the second optical fiber.

11. A tissue interface pad for applying an optical signal to tissue of a patient, the tissue interface pad comprising:
a first surface configured to interface with the tissue of the patient;
at least one guide channel disposed within the tissue interface pad and configured to route an input optical fiber carrying the optical signal to a first location in the tissue interface pad; and
a second surface configured to reflect the optical signal received from the input optical fiber through an optically transmissive portion of the tissue interface pad to direct the optical signal into the tissue through the optically transmissive portion of the tissue interface pad and the first surface.

12. The tissue interface pad of claim 11, wherein the optically transmissive portion of the tissue interface pad is index matched to the input optical fiber and optically mated to an end of the input optical fiber at the first location.

13. The tissue interface pad of claim 11, wherein an end of the input optical fiber comprises a generally flat third surface perpendicular to a longitudinal axis of the input optical fiber, and wherein the third surface is optically mated to a generally flat fourth surface of the tissue interface pad at the first location.

14. The tissue interface pad of claim 13, wherein the second surface is configured to direct the optical signal perpendicular to the longitudinal axis of the input optical fiber to direct the optical signal through the optically transmissive portion of the tissue interface pad and the first surface.

15. The tissue interface pad of claim 11, further comprising a cladding portion coupled to the second surface, wherein the optically transmissive portion of the tissue interface pad comprises a first optical index material, and wherein the cladding portion comprises second optical index material with an optical index lower than the first optical index material.

16. A tissue interface pad for applying an optical signal to tissue of a patient, the tissue interface pad comprising:
a first surface configured to interface with the tissue of the patient;
at least one guide channel disposed within the tissue interface pad and configured to route an input optical fiber carrying the optical signal to a first location in the tissue interface pad;
an optical interface element coupled to one end of the input optical fiber at the first location and configured to direct the optical signal received from the input optical fiber toward a second surface; and
the second surface configured to reflect the optical signal to direct the optical signal into the tissue through the optical interface element and the first surface.

17. The tissue interface pad of claim 16, wherein the optical interface element comprises an optical adhesive configured to optically mate the end of the input optical fiber to the second surface, and wherein the second surface comprises an optically reflective surface.

18. The tissue interface pad of claim 17, wherein the at least one guide channel comprises a chamber at the first location configured to hold the optical adhesive, and wherein the optical adhesive is disposed in the chamber to form a prism comprising the optical adhesive.

19. The tissue interface pad of claim 16, wherein the second surface comprises an optically reflective and angled surface configured to reflect the optical signal generally perpendicular to a longitudinal axis of the input optical fiber to direct the optical signal through the optical interface element and the first surface.

20. The tissue interface pad of claim 16, wherein the optical interface element comprises an angled cut in the input optical fiber to form a hypotenuse on the end of the input optical fiber, wherein an outer surface of the hypotenuse comprises the second surface, and wherein the outer surface is coated by a reflective material.
